(19) United States
(12) Patent Application Publication
(10) Pub. No.: US 2026/0337659 A1
(43) Pub. Date: Oct. 29, 2026

(54) DETECTING CONTAMINANTS DURING THERAPY DEVELOPMENT AND MANUFACTURE

(71) Applicant: National Resilience, LLC, Blue Ash (00)

(72) Inventors: Neeraj Salathia, San Diego, CA (US); Sophia Espanola, San Diego, CA (US); Saege Hancock, San Diego, CA (US); Elizabeth Lamarca, San Diego, CA (US); Dwight Baker, San Diego, CA (US); Ekta Dadlani, San Diego, CA (US); Fan Yang, San Diego, CA (US); Dave Robbins, San Diego, CA (US); Tierney Bougie, San Diego, CA (US); Hana Ha, San Diego, CA (US); Sonal Choudhary, San Diego, CA (US); David Conant, San Diego, CA (US); Nicholas Orangio, San Diego, CA (US); Jordan Uyeki, San Diego, CA (US); Tahani Al Bulushi, San Diego, CA (US)

(21) Appl. No.: 19/700,011

(22) Filed: Jun. 5, 2026

Related U.S. Application Data
(63) Continuation of application No. PCT/US2024/059027, filed on Dec. 6, 2024.
(60) Provisional application No. 63/606,823, filed on Dec. 6, 2023.

(51) Int. Cl. C12Q 1/6806; C12N 5/00

(52) U.S. Cl. CPC C12Q 1/6806; C12N 5/0081

(57) ABSTRACT

This disclosure provides methods, systems, and kits for detecting contaminants during manufacture of therapeutic compositions, including obtaining a sample including production cells and potential contaminants; removing one or more production cells from the sample to generate a production cell-depleted sample; selectively reducing production cell nucleic acids from the production cell-depleted sample to produce a contaminant nucleic acid-enriched sample; amplifying contaminant nucleic acids using one or more nucleic acid primers that selectively hybridize with one or more conserved genomic nucleic acid sequence regions of the potential contaminants to produce an amplified contaminant nucleic acid sample; sequencing the amplified contaminant nucleic acid sample to produce a plurality of reads; and determining, using an abundance estimation, a quantified contaminant taxa abundance profile of the amplified contaminant nucleic acid sample.

### CROSS-REFERENCE TO RELATED APPLICATIONS

This application is a Continuation Application, and claims benefit of, International Application No. PCT/US24/59027, filed on Dec. 6, 2024, which claims the benefit of U.S. Provisional Patent Application No. 63/606,823, filed on Dec. 6, 2023, which is incorporated herein by reference in their entirety.

### TECHNICAL FIELD

This disclosure relates to methods and systems for detecting contaminants during the development and/or manufacture of therapeutics, e.g., using Next Generation Sequencing (NGS). For example, the methods and systems described herein can be used to detect microbial contaminants.

### BACKGROUND INFORMATION

Maintaining the sterility of cell culture is an important aspect in the development and/or manufacture of cell-based and gene-based therapies, as contamination can compromise the safety and efficacy of the final products. Cell culture techniques can be employed for the detection of cellular contaminants, as they offer the capacity to amplify the presence of contaminating cells or microorganisms. Through testing and analysis, such as microscopic examination and growth-based methods, contaminants can be identified and quantified, enabling the implementation of measures to prevent contamination.

### SUMMARY

This disclosure provides methods and systems for detecting contaminants during therapy development and/or manufacture. Microorganism contaminants are often present in low numbers and can be associated with factors such as poor cell viability and challenges associated with cell culture conditions. The methods and systems described herein address limitations in contaminant detection that are unmet by current technologies, increase contamination signals detected via Next Generation Sequencing, and utilize bioinformatics techniques to identify contaminants at the genus and/or species level. If such contaminants are detected in a therapeutic composition, the composition can be destroyed as being unsafe.

In general, the disclosure features methods for detecting contaminants during manufacture of a therapeutic composition, the methods including or consisting of: (a) obtaining from a liquid used in the manufacture of the therapeutic composition a sample comprising production cells and potential contaminants; (b) removing one or more production cells from the sample to generate a production cell-depleted sample; (c) selectively reducing a level of production cell nucleic acids from the production cell-depleted sample to produce a contaminant nucleic acid-enriched sample; (d) amplifying contaminant nucleic acids in the contaminant nucleic acid-enriched sample using one or more nucleic acid primers that selectively hybridize with one or more conserved genomic nucleic acid sequence regions of the potential contaminants to produce an amplified contaminant nucleic acid sample; (e) sequencing the amplified contaminant nucleic acid sample to produce a plurality of reads; and (f) determining, using an abundance estimation, a quantified contaminant taxa abundance profile of the amplified contaminant nucleic acid sample.

In some embodiments, the contaminant nucleic acid-enriched sample includes RNA. In some embodiments, the production cells are mammalian cells. In some embodiments, the mammalian cells are human cells. In some embodiments, some, most, substantially all, or all of the production cells are removed based on a cell surface marker, size, and/or density of the production cells. In some embodiments, the cell surface marker is or includes a protein. In some embodiments, the surface marker, e.g., a cell surface receptor, includes one or more of CD3, CD4, CD8, CD28, CD41, CD45, CD62, CD138, CD235a, CD14, CD123, PD-1, ID1, CTLA-4, CD19, CD20, CD22, CD56, CD16, CD14, CD15, CD66, or CD34.

In some embodiments, reducing the level of production cell nucleic acids includes removing at least 90% of the production cell nucleic acids. In some embodiments, the production cell nucleic acids comprise DNA or RNA.

In certain embodiments, the potential contaminants are one or more of a fungus, a yeast, a bacterium, a virus, or a mycoplasma. In some embodiments, at least one of the potential contaminants is a mycoplasma. In some embodiments, the sample is a cell culture sample, and the plurality of production cells are a plurality of mammalian T-cells.

In some embodiments, prior to step (d), the methods further include lysing cells remaining in the production cell-depleted sample. In some embodiments, step (c) includes inhibiting amplification of a particular gene using a complementary oligonucleotide that prevents reverse transcription of the particular gene. In certain embodiments, the complementary oligonucleotide are or include a locked nucleic acid (LNA) that is complementary to the particular gene. In some embodiments, the particular gene is a human beta actin gene. In some embodiments, the particular gene includes one or more genes that encode human cytoplasmic rRNA 5S, 5.8S, 18S, and 28S; human mitochondrial rRNA 12S and 16S; and human mitochondrial rDNA ND1, ND2, ND3, ND4, ND 4L, ND5, ND6, COX1, COX2, COX3, ATP6, and CYTB. In some embodiments, the one or more conserved genomic nucleic acid sequence regions of the potential contaminants are one or more of a 16S, 18S, 25S, ITS1, or 28S ribosomal region of the potential contaminants.

In certain embodiments, step (d) further includes generating an NGS library with the amplified contaminant nucleic acids. In some embodiments, step (d) further includes using random hexamers with the one or more nucleic acid primers that selectively hybridize with the one or more conserved genomic nucleic acid sequence regions of the potential contaminants to produce a cDNA and amplified contaminant nucleic acid sample. In some embodiments, prior to step (d), the method further includes applying one or more intercalating dyes to the sample.

In some embodiment, the abundance estimation is performed using a differential abundance algorithm. In some embodiments, step (f) further includes: comparing the plurality of reads from the amplified contaminant nucleic acid sample to a database of microbial genomes; identifying one or more contaminant taxa, based on a comparison of a plurality of reads of the amplified contaminant nucleic acid sample to the database of microbial genomes; sequencing a negative control to produce a plurality of reads from the negative control; comparing the plurality of reads from the negative control to the database of microbial genomes; and identifying one or more contaminant taxa in the sample, based on a comparison of results from the negative control to results from the sample.

In some embodiments, the methods further include: determining, using the abundance estimation of the plurality of reads of the negative control, a quantified contaminant taxa abundance present in the negative control; comparing the quantified contaminant taxa abundance present in the amplified contaminant nucleic acid sample to the quantified contaminant taxa abundance present in the negative control; and determining one or more significantly abundant contaminant taxa in the amplified contaminant nucleic acid sample based on the quantified contaminant taxa abundance present in the amplified contaminant nucleic acid sample and the quantified contaminant taxa abundance present in the negative control.

In some embodiments, the methods further include identifying significantly abundant contaminant taxa by using machine learning, e.g., in as few as one sample. In some embodiments, the machine learning is trained based on an abundance distribution of read counts, unique molecular identifiers (UMIs), estimated true UMIs, or other normalized counts of individual microorganisms identified in each sample. In some embodiments, the method further comprises using a pool of sequences simulated from a database to determine a likelihood of false identification of each microorganism via repeated sequence alignments to provide a baseline of alignment errors for each microorganism.

In certain embodiments, the methods further include using a Z-score method to determine significance of the detected microorganisms based on their abundances. In some embodiments, the method further comprises using a pool of sequences simulated from a database to determine a likelihood of false identification of each microorganism via repeated sequence alignments to provide a baseline of alignment errors for each microorganism. In some embodiments, the method further comprises destroying the therapeutic composition if any contaminant is detected in the sample. In some embodiments, the methods further include filtering or masking of reads containing gene-specific primer sequences and/or location-specific sequences adjacent to a priming location.

In some embodiments, prior to step (f), the methods further include aligning the plurality of reads from the amplified contaminant nucleic acid sample to a database of microbial genomes; determining an alignment quality of respective reads of the plurality of reads of the amplified contaminant nucleic acid sample; and omitting respective reads of the plurality of reads from the amplified contaminant nucleic acid sample when the alignment quality of the respective reads is below a threshold. In some embodiments, the database is derived from public, private, and/or internally derived genomic sequences targeted to rRNA regions or is a “chopped” data base of gene-specific primer regions of genomic sequences.

In general, the disclosure further features hardware devices and systems, including a system that includes a hardware device, comprising a non-transitory computer-readable storage medium coupled to one or more processors and having instructions stored thereon which, when executed by the one or more processors, cause the one or more processors to perform operations any one of the methods described herein.

In another aspect, the disclosure features kits, including one or more reagents corresponding to any of the methods and processes described herein. In some embodiments, the one or more reagents are designed for cell depletion, removal of free-flowing nucleic acids, lysis, RNA purification, human RNA depletion, cDNA amplification of contaminants, or for RNA library construction, and/or reagents for library quality control. In some embodiments, the one or more reagents include one or more primers. In some embodiments, the reagents designed for RNA depletion include one or more of platinum-based compounds, e.g., platinum (II) chloride, tetrakis (triphenylphosphine) platinum), palladium-based compounds, e.g., diamminedichloro palladium (II), palladium (II) acetate, and/or ethidium monoazide (EMA) and propidium monoazide (PMA).

In another aspect, the disclosure also features methods of amplification of fragments whereby contaminant sequences are optimized for their number of base pairs. These methods include selecting specific primer oligonucleotide sequences and manipulating reaction conditions, wherein optimization of these conditions enhances contaminant detection and sequence-based identification of contaminant genus and/or species.

The methods described herein provide advantages over current methods of contaminant detection. In particular, the highly sensitive methods and systems described herein can be used to identify contaminants present at very low levels and enable more rapid detection than current culturing methods, as well as provide more detailed information, at both the genus and species level. For example, embodiments described herein can be used to identify a large variety of contaminants with increased efficiency and speed. The described methods can increase efficiency, for example, by reducing the need for multiple culture media for different classes of contaminating organisms.

As used herein, the term “contaminant” includes an element that is not part of the therapy development and manufacture. Examples of contaminants include microbial contaminants, such as bacteria, virus, fungus, and/or mycoplasma, and contaminants also include contaminating cells (e.g., cell lines that contaminate production cell lines).

As used herein, the term “production cells” include cells used during therapy development and manufacture, such as mammalian cells (e.g., human cells, non-human primate cells, canine cells, rabbit cells, porcine cells, and rodent cells), yeast cells, insect cells, and bacterial cells.

Unless otherwise defined, all technical and scientific terms used herein have the same meaning as commonly understood by one of ordinary skill in the art to which this disclosure pertains. Although methods and materials similar or equivalent to those described herein can be used to practice the invention as claimed, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention as claimed are set forth in the accompanying drawings and the description below. Other features, objects, and advantages of the invention as claimed will be apparent from the description and drawings, and from the claims.

### DESCRIPTION OF THE DRAWINGS

FIG. 1 is a flow chart of an example of a method as described herein for detecting contaminants during therapy development and/or manufacture.

FIG. 2 is a flow chart of an example of a wet lab workflow and a bioinformatics workflow for detecting contaminants during therapy development and/or manufacture, as described herein.

FIG. 3 is a flow chart of an example of a workflow for detecting contaminants during therapy development and/or manufacture, as described herein.

FIGS. 4A and 4B are flow charts of examples of a differential abundance-based statistical testing workflow for detecting contaminants during therapy development and/or manufacture, as described herein.

FIG. 5 is a flow chart of another example of a bioinformatics workflow for detecting contaminants during therapy development and/or manufacture, which includes unclassified reads, as described herein.

FIG. 6 is a schematic diagram of a system for detecting contaminants during therapy development and/or manufacture, as described herein.

FIG. 7 is a bar graph of positive selection of mycoplasma cells as described in Example 1 according to methods described herein.

FIGS. 8A and 8B are graphs of human ribosomal depletion as described in Example 2 according to methods described herein.

FIG. 8C is a bar graph of a proportion of spiked mycoplasma cells at levels.

FIG. 8D is a bar graph of a reduction in transcript signal of beta actin.

FIGS. 9A and 9B are bar graphs of gene specific amplification of adventitious agents as described in Example 3 according to methods described herein.

FIG. 10 is a bar graph of chemical reduction of signal originating from dead cells and contaminating DNA as described in Example 4 according to methods described herein.

FIG. 11 is a graph of a process control signal as described in Example 5 according to methods described herein.

FIG. 12 is a series of bar graphs showing the effectiveness of negative control in reducing background as described in Example 6 according to methods described herein.

FIG. 13 is a graph showing that machine learning trained with a cumulative distribution of detected taxa can be used to differentiate true contaminants and background taxa, when no negative controls are provided.

### DETAILED DESCRIPTION

The methods and systems disclosed herein can be used for the detection of contaminants that may be present during the development and/or manufacture of cell-based and/or gene-based therapeutic compositions, and overcome problems found in other methods of contaminant detection. The methods and systems disclosed herein use a unified approach that combines multiple different assays to increase contamination signal detected via NGS and bioinformatics techniques to determine a contaminant at the genus and/or species level.

In one aspect, the assays described herein include one or more of production cell depletion (e.g., human cell depletion), gene-specific cDNA amplification of pathogens, probe-based sequence depletion, chemical reduction of signal originating from dead cells and/or contaminating DNA, RNA library preparation and RNA sequencing, and bioinformatics analysis.

Non-limiting examples of cell-based and/or gene based therapeutic compositions include autologous cell therapy (e.g., CAR-T-cell therapy), allogeneic cell therapy (e.g., mesenchymal stem cell therapy), induced pluripotent stem cell (iPSC) therapy (e.g., reprogramming adult cells to pluripotent stem cells e.g., iPSC-derived retinal cells), gene editing therapies (e.g., CRISPR), in vivo gene therapy (e.g., vector functional gene delivery), ex vivo gene therapy (e.g., patient cells genetically modified in the lab and then returned to the patient).

Various types of samples can be tested for contaminants that may be present during the development and/or manufacture of cell-based and/or gene-based therapeutic compositions. For example, samples can include reagents used in cell culture and/or sequencing, such as growth media, antibiotics, antifungal agents, cryopreservatives, buffering agents, enzymes like DNA polymerase and reverse transcriptase, primers, dNTPs, viral vectors, transfection reagents, serum, cytokines, chemokines, detergents, co-factors, inducers like IPTG or doxycycline, fluorescent markers, stabilizers, fixatives, protease inhibitors, chelating agents, reducing agents, ethanol, isopropanol, formaldehyde, and methanol. In some embodiments, when samples do not include mammalian cells, some steps of the method are omitted (e.g., the cell depletion step can be omitted).

### Methods

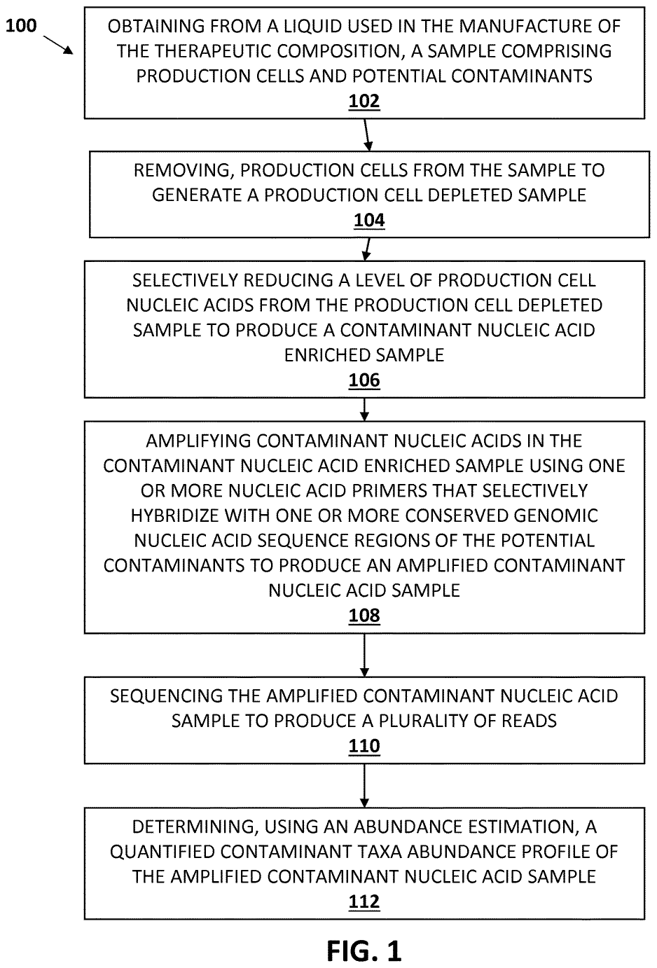

FIG. 1 depicts an example of a method 100 that is executed in accordance with implementations of the present disclosure to detect contaminants that may be present during the development and/or manufacture of cell-based and/or gene-based therapeutic compositions. In some embodiments, method 100 is performed by one or more computers. For example, a hardware device that manipulates reagents to execute one or more of workflows. In some embodiments, the method is implemented in the hardware device by digital electronic circuitry, or in computer hardware, firmware, software, or in combinations of them. The method 100 can be implemented in a computer program product tangibly embodied in an information carrier (e.g., in a machine-readable storage device) for execution by a programmable processor; and the method steps can be performed, for example, by a programmable processor executing a program of instructions to perform functions.

Method 100 includes step 102, obtaining from a liquid used in the manufacture of the therapeutic composition, a sample comprising production cells/drug product and potential contaminants. In some embodiments, a sample also includes reagents (e.g., cell culture reagents), plasmids, viral vectors, or other particles used in therapy development and manufacture. In some embodiments, the sample is a cell culture sample, e.g., from a therapeutic product and/or an in-process sample containing cells (e.g., mammalian cells). In some instances, the mammalian cells are human cells. In some instances, the therapeutic product does not contain any cells.

In some embodiments, the concentration of cells in the sample is about 0 cells/mL to about 1×10⁸ cells/mL. For example, the concentration of cells in the sample can be about 1×10⁵ cells/mL to about 1×10⁷ cells/mL. In some incidents, the concentration of cells in the sample are about 1×10⁶ cells/mL. Samples can include any one or more of reagents e.g., cell culture media, dimethyl sulfoxide, serum (e.g., serum albumin), polysaccharides (e.g., dextrose), sodium chloride, growth media, antibiotics, antifungal agents, cryopreservatives, buffering agents, viral vectors, transfection reagents, cytokines, chemokines, detergents, co-factors, and stabilizers. The volume of the sample can be about 0.1 mL to about 10 mL, based on user configurations.

A control sample includes a sample to be tested and reagents used to detect contaminants. In some embodiments, a control sample is processed at the same time or at about the same time as the sample. For example, a control sample, including the same reagents used with the sample, is processed through the workflow in parallel with the sample. In such examples, the analysis compares a potential contaminant taxon in both the control and the cell culture sample to assess the reliability of the detection method and determine contaminants potentially present in reagents.

Method 100 continues at step 104 by removing production cells from the sample to generate a production cell-depleted sample. Various strategies can be used to deplete cells for contaminant detection, as described herein, including filtration using membrane discs, membrane cell strainers, syringes, cellulose paper discs (e.g., having pore sizes ranging from 3-12 micron), a microfluidic cell shorting chip, and/or antibody selection. In some embodiments, centrifugation by density exclusion is used. In such embodiments, the filtration and density exclusion are implemented, e.g., at room temperature or higher, and, for example, at a slightly alkaline pH (e.g., from 7.0-8.0). In some embodiments, a microfluidic spiral chip is used to passively separate cells and particles according to their size based on the Dean forces. In some embodiments, the cell depletion is antibody-based cell depletion where cells are depleted based on a cell surface protein.

In other embodiments, a differential lysis of human and/or animal cells through osmotic/cytolysis lysis (e.g., saponin) with or without pretreatment with an enzyme (e.g., Proteinase K) and subsequent enzymatic (e.g., Benzonase, Decontaminase™, RNaseA, MNase) digestion or inactivation of human/animal nucleic acid is implemented. In such examples, incubation is performed, e.g., at room temperature or up to about 37 °C, and, in some embodiments, at slightly alkaline pH (e.g., from 7.0-7.6).

Method 100 continues at step 106 by selectively reducing a level of production cell nucleic acids from the production cell-depleted sample to produce a contaminant nucleic acid-enriched sample. The techniques employed are aimed at selectively reducing the presence of production cell nucleic acids within the production cell depleted sample to effectively lower background noise and increase a signal originating from potential contaminants.

In some embodiments, selectively reducing a level of production cell nucleic acids includes removing free-floating nucleic acids from the sample. Examples of free-floating nucleic acids are DNA or RNA molecules that are not contained within cells or encapsulated by membranes (also referred to as cell-free DNA or cell-free RNA). The presence of free-floating nucleic acids in a sample contributes to background noise in downstream analysis, making it challenging to detect signal from potential contaminants. The dilution of signal from potential contaminants reduces the assay's sensitivity and specificity, leading to less accurate results. In some embodiments, selectively reducing a level of production cell nucleic acids include a depletion of DNA (e.g., via DNase treatment) or RNA (e.g., ribosomal RNA (rRNA), mitochondrial RNA (mtRNA)) from the production cells. For example, human rRNA/mtRNA can be depleted from the sample and/or control using a magnetic bead assay that is coated with probes complementary to human rRNA sequences and/or using a locked nucleic acid oligonucleotide that is complementary to human rRNA sequences. In some embodiments, the sample does not include cells. In such examples, the selective reduction of cell lines can be ignored.

Method 100 continues at step 108 by amplifying contaminant nucleic acids in the contaminant nucleic acid enriched sample using one or more nucleic acid primers that selectively hybridize with one or more conserved genomic nucleic acid sequence regions of the potential contaminants to produce an amplified contaminant nucleic acid sample. These regions can include, but are not limited to, 5S, 16S, 18S, 23S, 28S, and ITS conserved regions and/or random base primers (i.e., random hexamer). After depleting the sample of production cells and reducing a level of production cell nucleic acid, reverse transcription is performed to convert potential contaminant RNA into cDNA. Primers and probes are designed to target bacteria, fungi, virus, and/or mycoplasma generally, and/or specifically.

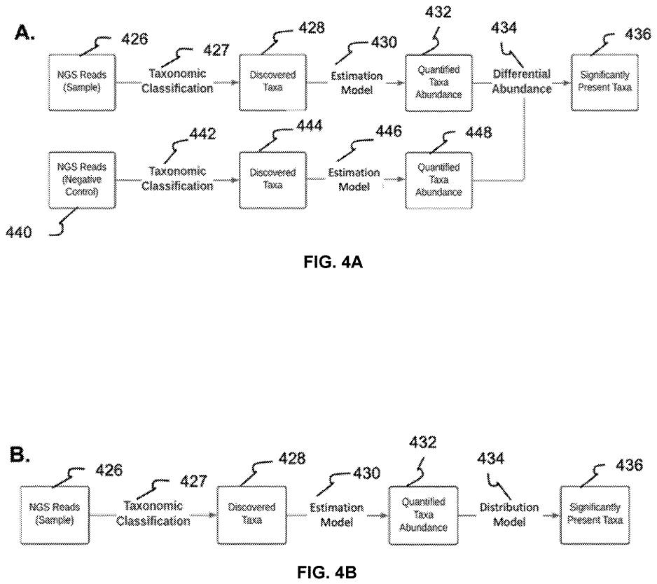

Method 100 continues at step 110 by sequencing the amplified contaminant nucleic acid sample to produce a plurality of reads. In some embodiments, sequencing (e.g., NGS) is executed in a separate device as the other steps of the workflow. In such examples, the amplified contaminant nucleic acid sample is sequenced in a sequencer to produce a plurality of reads. In some embodiments, sequencing takes place after RNA library construction and library QC of the amplified contaminant nucleic acid sample. The reads generated by sequencing the amplified contaminant nucleic acid sample and/or the control are imported via one or more computers, to a hardware device for a bioinformatics workflow. Method 100 continues at step 112 by determining, e.g., using an abundance estimation, a quantified contaminant taxa abundance profile of the amplified contaminant nucleic acid sample. In some embodiments, the quantified taxa abundance of the amplified contaminant nucleic acid sample and control is estimated using a Bayesian estimation model. For example, an abundance estimation includes (1) alignment of reads to a database of genomes corresponding to the contaminants of interest, and (2) estimating abundance using a taxonomic-based Bayesian estimation model. In such examples, the model computes a probability distribution for the abundance of each taxon, offering a measure of uncertainty along with the estimate. The abundance of each discovered taxa is compared to its equivalent abundance in the negative control via a differential abundance analysis, allowing the assay to identify contaminating taxa that are specific to the sample. Step 112 is described in further detail in connection with FIGS. 4A-4B.

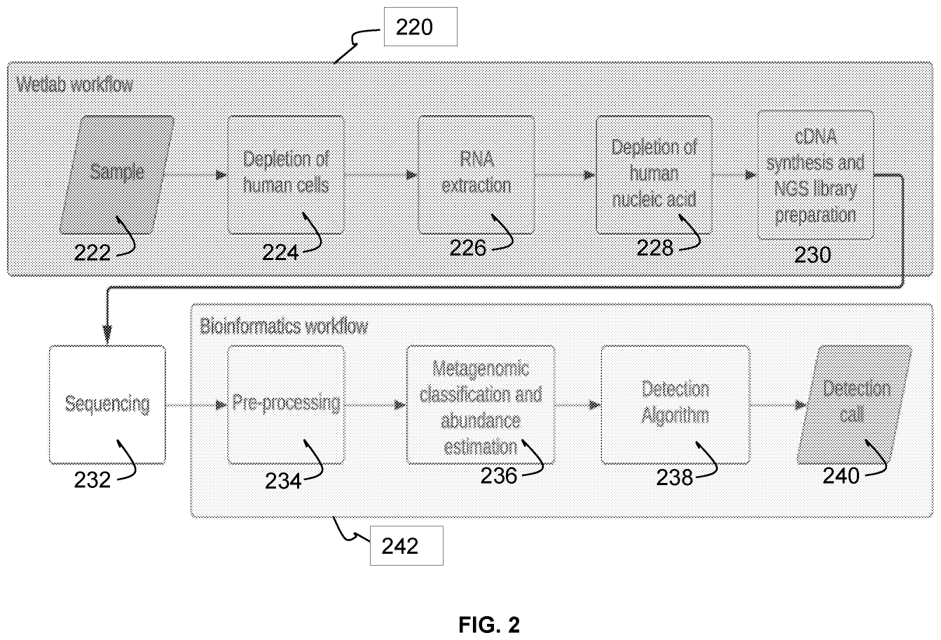

FIG. 2 is a flow chart of an example of a wet lab workflow 220 and a bioinformatics workflow 242 for detecting contaminants during therapy development and/or manufacture, as described herein. The steps of the example wet lab workflow 220 and the example bioinformatics workflow 242 are executed within a hardware device as described herein. For example, a hardware device is equipped with software and/or hardware accessible to users through a user-interface and operable to retrieve stored instructions that when executed by one or more processors can perform steps for the wet lab workflow 220. The reagents for the wet lab workflow 220 are either preloaded in the device or available in compatible cartridges.

For example, a hardware device can include a port or external access to provide a sample 222 comprising production cells and potential contaminants and/or a control. Reagents are included in the hardware device or in compatible cartridges for one or more steps of the wet lab workflow 220 e.g., depletion of human cells 224, RNA extraction 226, depletion of human nucleic acids 228, and cDNA synthesis and NGS library prep 230.

For example, devices and/or reagents for cell depletion 224 to produce a production cell-depleted sample include filtration reagents e.g., membrane discs, membrane cell strainers, syringes, cellulose paper discs, and/or microfluidic cell/particle separation chips, reagents for differential lysis, and reagents for antibody-based cell depletion. In embodiments of wet lab workflows that include RNA extraction 226, reagents for the extraction of RNA from potential contaminants are included, e.g., reagents for magnetic bead RNA extraction and reagents for centrifugation-based RNA extraction. In embodiments where a level of production cell nucleic acids is removed to produce a contaminant nucleic acid enriched sample, reagents for depletion of human nucleic acids 228 are included. For example, magnetic beads coated with probes complementary to human rRNA sequences can be included in the hardware device and/or compatible cartridges. The example of the wet lab workflow chart 220 can include amplifying contaminant nucleic acids in the contaminant nucleic acid enriched sample. For example, the hardware device can include reagents for the cDNA synthesis and NGS library preparation at step 230. In such examples, the reagents include one or more nucleic acid primers that selectively hybridize with one or more conserved genomic nucleic acid sequence regions of the potential contaminants to produce an amplified contaminant nucleic acid sample.

In some embodiments, sequencing of the amplified contaminant nucleic acid sample is performed in a separate device. For sequencing 232, the amplified contaminant nucleic acid sample is removed from the hardware device and sequenced in a sequencing device. After sequencing, the reads generated by sequencing the amplified contaminant nucleic acid sample and/or the control are imported, via one or more computers, back to the hardware device, or to a different hardware device, for the bioinformatics workflow 242.

The generated reads are imported to the hardware device. The hardware device is equipped with software and/or hardware accessible to users through a user-interface and operable to retrieve stored instructions that when executed by one or more processors can perform steps for the bioinformatics workflow 242. The example bioinformatics workflow 242 includes pre-processing 234. Pre-processing includes separating low-quality reads, low-complexity reads, short-length reads identified as originating from a production cell. In some examples, the separated reads are saved for further analysis. Metagenomic classification and abundance estimation 236 includes (1) alignment of reads to a database of genomes corresponding to the contaminants of interest, and (2) estimating abundance using a taxonomic-based detection algorithm. In some embodiments, the metagenomic classification and abundance estimation 236 further includes read normalization, wherein the read normalization can be performed according to library depth or composition. In some embodiments, read normalization can include counts per million (CPM) normalization or Trimmed Mean of M-values (TMM) normalization, e.g., employing a Bayesian estimation model.

The detection algorithm 238 detects differentially abundant taxa identified between the sample and a control sample to result in a detection call 240 for a contaminant. This includes assessing the differential abundance and identifying significantly present taxa. For example, this determination includes utilizing an estimation algorithm for reads from the negative control to quantify the abundance of contaminant taxa in the negative control. Subsequently, a comparison is made between the quantified contaminant taxa abundance in the amplified contaminant nucleic acid sample and that in the negative control, leading to the identification of one or more significantly present taxa in the amplified contaminant nucleic acid sample based on these abundance comparisons. Additionally, the algorithm incorporates the optionality to not use a negative control and utilize an internal cumulative distribution model or a synthetically derived contaminant sample distribution model for contaminant taxa identification.

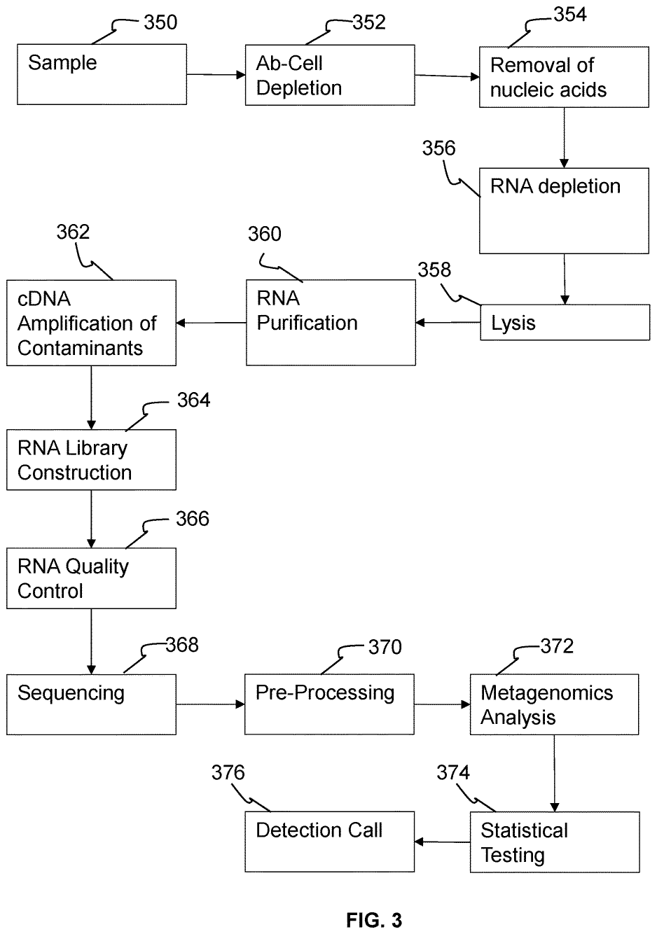

FIG. 3 is a flow chart of an example of an embodiment of a workflow for detecting contaminants during therapy development and/or manufacture, as described herein. In a first step 350, a sample is obtained for a workflow for detecting potential contaminants. In some embodiments, a volume of sample is about 2 mL and can include reagent components. For example, a cell culture sample can include components such as antibiotics, growth media, dextrose, and cryopreservation agents such as DMSO. In some embodiments, the sample is a solid (e.g., a pellet of cells) that is reconstituted. In such examples, the contaminant detection workflow is applied just as it would be for a liquid sample. In parallel, a known non-contaminated control (e.g., non-contaminated cell culture or reagents) is processed through each step of the workflow from preparation to data analysis. This control aids in validating the accuracy of the workflow, as any discrepancies in identified contaminants can highlight issues or confirm the significance of the detected contaminants. Another control that can be used can include a known contaminant at a known concentration.

The next step 352 is cell depletion, e.g., antibody-based cell depletion. For example, production cells are removed from the sample and/or the control at step 352 to produce a production cell-depleted sample. The production cells are selectively removed based on a cell surface marker. For example, antibody-based cell depletion includes the depletion of production cells based a cell surface protein (e.g., one or more of CD3, CD4, CD8, CD28, CD41, CD45, CD62, CD138, CD235a, CD14, CD123, PD-1, ID1, CTLA-4, CD19, CD20, CD22, CD56, CD16, CD14, CD15, CD66, or CD34). Strategies for antibody-based cell depletion include complement-mediated lysis, magnetic separation (e.g., antibodies are conjugated with magnetic beads, allowing magnetic separation of tagged cells), and/or flow cytometry, e.g., using fluorescently labeled antibodies. A plurality of cells are depleted from the sample to generate the cell-depleted sample. For example, about 80% of production cells to about 100%, e.g., 85, 90, 95, 96, 97, 98, 99, or 100% of production cells are depleted during the antibody-based cell depletion. In step 354, optionally, removal of production cell nucleic acids can include the removal of free-floating nucleic acids. Strategies for removing production cell nucleic acids include enzymatic (e.g., Benzonase, Decontaminase™, RNaseA, MNase, DNase, etc.) and bead-based (e.g., charge-switch, cationic, and/or Poly-A coated) methods. Free-floating nucleic acids can be inactivated, for example, by cross-linking through cytotoxic methods for downstream molecular based-assays using platinum-based compounds (e.g., platinum (II) chloride, tetrakis (triphenylphosphine) platinum), palladium-based compounds (diamminedichloro palladium (II), palladium (II) acetate), and/or ethidium monoazide (EMA) and propidium monoazide (PMA). Inactivated free-flowing nucleic acids will not cause background as they cannot be sequenced.

In step 356, optionally, selectively reducing a level of production cell nucleic acids from the production cell-depleted sample can include exclusion or blockage of RNA (rRNA and/or mtRNA) from the cells being used in the sample (e.g., mammalian or yeast RNA). For example, RNA is excluded using methods that target mammalian rRNA and/or mtRNA (e.g., HMR rRNA/mtRNA). The selective blockage of mammalian rRNA excludes non-contaminant nucleic acids thereby enriching the contaminant nucleic acids of the sample (e.g., producing a contaminant nucleic acid-enriched sample). For example, techniques for RNA exclusion can include bead-based methods, mammalian mtRNA methods, inhibition of reverse transcription, and/or PCR amplification using locked nucleic acids (LNA) (e.g., human beta actin), and/or single-stranded DNA probes in conjunction with RNase H/DNase I digestion. In other examples, RNA is depleted using CRISPR-based methods to target and cleave rRNA.

In some embodiments, RNA (rRNA and/or mtRNA) depletion from the sample can include the use of magnetic beads coated with nucleic acid probes complementary to RNA sequences. The sample can be mixed with the beads to allow for hybridization between the RNA and the complementary nucleic acid probes on the beads. This hybridization facilitates the specific capture of RNA molecules. After incubation, the bead-bound RNA is separated from the rest of the RNA sample using a magnetic field. By discarding the magnetic bead fraction, a majority of the non-target RNA is removed from the sample. The remaining RNA fraction, now enriched for messenger RNA (mRNA) and/or other RNA of the potential contaminant of interest, are eluted and prepared for downstream applications.

In step 358, contaminant cells in the sample are lysed. Lysis techniques can include mechanical lysis, which includes the release of DNA and RNA from contaminant cells, for example, using silica or ceramic beads (with a bead size that can range from about 100 microns to about 800 microns). Other bead materials include glass, zirconium silicate, zirconium oxide, silicon carbide, and stainless steel. The lysis reaction can be carried out in tubes, e.g., of about 1 mL to about 2 mL, e.g., placed on a rapid shaker with a speed range of 2700-3200 RPM for about 1 minute to about 20 minutes (e.g., about 5 minutes to about 20 minutes, about 10 minutes to about 20 minutes, about 1 minute to about 15 minutes, about 5 minutes to about 15 minutes, about 10 minutes to about 15 minutes, about 1 minute to about 10 minutes, about 5 minutes to about 10 minutes, or about 1 minute to about 5 minutes). In some embodiments, the lysis reaction is carried out in 0.5 mL to 2 mL chambers with a self-contained micromotor-based bead beater powered between about 2 volts to about 7 volts (e.g., about 4 volts to about 7 volts, about 6 volts to about 7 volts, about 2 volts to about 6 volts, about 4 volts to about 6 volts, or about 2 volts to about 4 volts) for about 1 minute to 20 minutes (e.g., about 5 minutes to about 20 minutes, about 10 minutes to about 20 minutes, about 1 minute to about 15 minutes, about 5 minutes to about 15 minutes, about 10 minutes to about 15 minutes, about 1 minute to about 10 minutes, about 5 minutes to about 10 minutes, or about 1 minute to about 5 minutes). Lysis procedures are performed at about room temperature (20-25 °C) or higher.

In step 360, the RNA in the sample is purified to increase the efficiency and effectiveness of RNA removal from the sample. RNA purification can involve isolating RNA molecules from the sample, e.g., using chemical reagents or columns to separate RNA from other cellular components. One example of a method for RNA purification includes acid phenol-chloroform extraction, in which RNA is separated from other cellular components such as DNA and proteins using a mixture of acid phenol and chloroform. After centrifugation, RNA is precipitated from the aqueous phase using alcohols like ethanol or isopropanol.

In a magnetic bead-based approach, RNA-binding magnetic beads are used to selectively capture RNA from a lysed cell sample. After binding, the beads are magnetically separated from the lysate, washed to remove contaminants, and the RNA is eluted. Both methods can include a DNase treatment step to remove any contaminating DNA. In another example, a purification protocol that includes an input volume ranging between 300-700 μL of lysis buffer is used. This lysis buffer can contain a reducing agent such as 1% β-mercaptoethanol or dithiothreitol to help break down cellular structures and release RNA. Alternatively, a buffer formulated for similar purposes can be employed. After combining the sample with the lysis buffer, the mixture is subjected to further processing steps, such as centrifugation or column-based separation to isolate the RNA for downstream applications.

In step 362, cDNA is prepared from the contaminant nucleic acid-enriched sample produced in steps 354 and/or 356 and amplified to produce an amplified contaminant nucleic acid sample. For example, reverse transcription is performed to convert the contaminant RNA into cDNA. The subsequent amplification, e.g., using the polymerase chain reaction (PCR), is tailored to selectively amplify this contaminant cDNA. The amplification significantly increases the sensitivity of the overall assay, enabling the identification of low-abundance levels of contaminants such as bacteria, virus, fungi, or mycoplasma that may be present in the sample.

Non-limiting examples of amplification techniques include the use of specifically designed primers and probes, the use of random hexamers, and the use of intercalating dyes. Intercalating dyes such as platinum chloride, propidium monoazide, and ethidium monoazide is used to chelate free-floating nucleic acids and inhibit their amplification. Primers and probes are designed to target bacteria, fungi, or mycoplasma. In some embodiments, conserved regions of contaminants are used as the basis or primer and/or probe design. For example, conserved regions such as 16S, 18S, 25S, internal transcribed spacer 1 (ITS1), and 28S ribosomal regions are targeted by primer and probe design. In some examples, random hexamers are used alongside the primers to capture organisms not covered by the gene-specific or conserved region primers.

In step 364, an RNA library is constructed. In some embodiments, after synthesizing the cDNA from RNA (e.g., in step 362), nucleic acid adapters are attached to both ends of the cDNA fragments. Adapters can allow the fragments to bind to the sequencing platform and enable the PCR that amplifies the material. Examples of adapters include Y-shaped, barcoded with unique molecular identifiers (UMIs), and indexed adapters. In some examples, adapters can facilitate the multiplexing of multiple samples in an individual sequencing run. Following the adapter attachment, size selection can occur to isolate cDNA fragments within a predetermined size range.

Non-limiting examples of methods for size selection include gel electrophoresis, column-based purification, and/or bead-based methods. In examples that utilize bead-based methods, the beads bind to the cDNA fragments and unwanted sizes are washed away, leaving behind cDNA of the predetermined size range. In this example, after size selection is complete, the adapter-ligated cDNA can undergo amplification through PCR generating a volume of cDNA for either sequencing or additional analysis.

RNA fragmentation time(s) (minutes) and NGS library bead-based cleanup steps can be manipulated to optimize the size (base-pairs) of the final NGS library insert, to facilitate robust nucleic acid sequence-based alignment, for species and genus microorganism contaminant identification.

The concentration of a library refers to the amount of prepared library molecules per unit volume. In some embodiments, libraries are first prepared in the high nanomolar (nM) range, normalized to low nM, and lastly diluted as a pool to mid-high picomolar (pM). For example, sample libraries described herein are prepared at a concentration higher than 0.5 nM. In some embodiments, sample libraries described herein are prepared at a concentration ranging from about 0.5 nM to about 3.0 nM. After library preparation, the libraries are diluted to a concentration suitable for sequencing. For example, the libraries are diluted to a concentration ranging from about 500 picomolar (pM) to about 1100 pM. In some examples, a known control (e.g., PhiX viral genome) is included in the sequencing run. For example, the control is included at about 1%-15% in proportion to the sequencing library.

In step 366, library quality control (QC) is done to assess the RNA library from step 364 for parameters including concentration and size/length. In some embodiments, an assessment of library concentration can utilize a fluorometric readout. For example, a working solution is prepared, and a standard curve is established for calibration of a fluorometer. In some instances, a diluted, known concentration of standards (e.g., a dsDNA standard) in the working solution is used to generate the standard curve. A small portion of the library generated in step 364, for example, is mixed with the working solution in a ratio of about 1:100 to about 1:300. An incubation period can follow for about 1 minute to about 7 minutes to allow a fluorescent dye to bind specifically to the dsDNA in the sample. After incubation, the fluorescence of the sample is measured using a fluorometer. In this example, the concentration of the DNA library is calculated based on these readings and the standard curve.

Another method for library quantification includes quantitative polymerase chain reaction (qPCR) to both evaluate library concentration and presence of adapters for sequencing. This embodiment is an example of a molecular based reaction that employs specific primers that can generate absolute, qPCR-based quantification through the use of standard curves. The assessment of the RNA library size can involve using capillary-electrophoresis and corresponding high sensitivity or DNA assays.

In step 368, the amplified contaminant nucleic acid sample produced in step 362 is sequenced to produce a plurality of reads. A sequencing protocol can start, for example, with a prepared DNA or cDNA library loaded onto a flow cell. A flow cell can have a surface that allows the library fragments to bind and form clusters (e.g., via bridge amplification). This results in one or more fragments generating clusters of sequences on the surface. Following cluster formation, sequencing reagents are introduced to initiate, for example, a sequencing-by-synthesis process. During this phase, fluorescently labeled nucleotides and a polymerase enzyme are added. The polymerase incorporates the nucleotides into the growing DNA strand, and each incorporation event is accompanied by the emission of a fluorescent signal.

In some embodiments, a camera or other detection system is used to capture fluorescent signals at each cycle of nucleotide incorporation. The color of the fluorescence indicates which nucleotide is being added to the growing DNA strand. By recording these signals in successive cycles, the sequence of each DNA fragment is determined. After the sequencing run, the raw data is processed to remove low-quality reads and to separate multiplexed samples, if applicable.

Read depth includes a number of sequencing reads generated for each sample. For example, roughly about 10 million reads to about 100 million reads (e.g., about 10 million reads to about 80 million reads, about 10 million reads to about 60 million reads, about 10 million to about 40 million reads, about 10 million reads to about 20 million reads, about 20 million reads to about 100 million reads, about 20 million reads to about 80 million reads, about 20 million reads to about 60 million reads, about 20 million to about 40 million reads, about 40 million reads to about 100 million reads, about 40 million reads to about 80 million reads, about 40 million reads to about 60 million reads, about 60 million reads to about 100 million reads, about 60 million reads to about 80 million reads, or about 80 million reads to about 100 million reads) is obtained for each sample. This range indicates the depth to which the sample's genetic material will be sequenced. Higher read depths can provide more comprehensive coverage of the genome or transcriptome. In some embodiments, the sequencing mode is paired-end mode with read lengths ranging from 100 base-pairs to 150 base pairs per read. For example, the paired-end mode is 2×, meaning that both the forward and reverse strands of each DNA fragment are sequenced separately. The read length indicates the number of bases that will be sequenced from each end of the DNA fragment.

In step 370, the sequence reads are preprocessed. For example, low-quality reads are separated from the main workflow based on a quality score (Q Score) cutoff (e.g., if >40% of a read has a Q score<15, the read pair is separated). Low complexity reads are separated from the main workflow based on a read entropy threshold (e.g., where reads with an entropy lower than 60 are considered low complexity and are removed). In some embodiments, adapter sequences and short-length reads are also removed. Production host cell reads are separated from the main workflow prior to downstream metagenomic analysis. At each step of pre-processing, separated reads are saved for further analysis.

In step 372, the workflow includes a downstream metagenomic analysis. Metagenomic analysis can include review of one or more databases established as known resources for comparison of contaminants. The database can initially include a wide range of genomes from various types of microorganisms, such as bacteria, fungi, and mycoplasmas. Such genomes are sourced from publicly available and commercially provided databases and can also include organisms specified in relevant regulatory guidelines or industry standards. In some examples, features of such databases include that they are modular and expandable. For example, when a manufacturing site or customer begins using the methods described herein, additional microbial genomes specific to their environment are incorporated into the existing database. This allows for a more customized and site-specific approach to microbial identification and analysis.

For example, a database can include a baseline of >10,000 unique microbial species obtained from publicly or privately available bacterial, fungal, and mycoplasma sequences, supplemented with organisms outlined in United States Pharmacopeia (USP) chapter 71 and USP chapter 63. The database is readily augmented with additional microbial genomes and/or genomic features (e.g., manufacturing site-specific isolates that are not in the provided databases are added to the database for use in the methods described herein during onboarding of a manufacture site and/or user).

In step 374, statistical tests are used to confirm the identification of the detected microorganisms. In some embodiments, statistical testing of the sequences compared to one or more controls are performed. For example, if one or more negative controls are included in the assay, an estimation algorithm (e.g., a differential abundance-based statistical testing method) is used to compare the microbial abundance profiles between the test sample and a negative control. If no negative controls are included, an alternative estimation algorithm is used to determine the threshold of background and contaminants. If one or more negative controls are provided, the microbial read abundance profiles, in terms of number of reads of each type of microorganism, is compared to negative controls using a statistical test based on a negative binomial generalized linear model. Non-limiting examples of statistical methods for differential abundance statistical tests include DESeq2, edgeR, limma baySeq, PoissonSeq, and Sleuth.

Alternatively in step 374, the algorithm incorporates the optionality to also use an internal cumulative distribution model (with unique molecular identifiers) or a synthetically derived contaminant sample distribution model in place of negative controls.

Step 376 provides a detection call, which indicates to the operator whether or not a contaminant is present in the sample. A detection call can include further information, such as the type or types of contaminants, the level or concentration of the contaminant(s) in the sample, and/or the genus and species of the contaminant(s).

FIGS. 4A-4B summarize the workflow of statistical testing step 374. FIG. 4A is a flow chart of an example of a differential abundance-based statistical testing workflow for detecting contaminants during therapy development and/or manufacture, as described herein. The example workflow of FIG. 4A, in some embodiments, can include one or more steps of the bioinformatics workflow 242 of FIG. 2. The bioinformatics workflow of FIG. 4A describes a differential abundance-based statistical testing method that is used to compare the microbial abundance profiles between a test sample and a negative control.

At step 426 the NGS reads of the sample can be obtained. The NGS high-throughput sequencing produces a large number of reads. For example, the sample can be a cell culture sample and the reads produced can include sequences representing the transcripts of contaminants present in the cell culture. In some embodiments, the reads are about 75 nucleotide bases to about 1000 bases in length, e.g., 100, 150, 200, 250, 300, 350, 400, 450, or 500 bases in length depending on the sequencing platform used. The reads can include coding regions, non-coding regions, and any mutations or variations specific to the contaminants. Reads can be either single or paired-end reads.

At step 440, NGS reads are also obtained from a negative control. For example, a negative control is processed with each step described herein. The negative control is one or more of a process control, sterile media, reagents, water, or cell culture. The reads produced can include sequences representing the transcripts of contaminants that were present initially in reagents shared with the sample or were picked up along the way through the wet lab workflow.

At step 427, the reads are analyzed for taxonomic classification. The resulting NGS reads undergo quality control to remove low-quality or ambiguous sequences. Further filtering or masking of reads may occur based upon reads containing the gene-specific primer sequences and proper location-specific sequence adjacent to the priming location. These cleaned reads can be aligned at step 428 against a reference database that contains known sequences from various taxa, using statistical testing designed for sequence matching. The abundance of unique molecular identifier (UMI) for each taxon is also determined at step 428. For example, if the focus is on bacterial DNA, each read that matches a particular bacterial species contributes to the evidence supporting the presence of this species in the sample. The frequency, type of matches, and UMI abundance can be used for the identification of organisms present. At step 442, the reads of the negative control are also filtered and masked as they are in step 427 and are analyzed for taxonomic classification. Cleaned control reads can be aligned, at step 444, against a reference database that contains known sequences from various taxa, using statistical testing designed for sequence matching. The abundance of UMI for each taxon in the control sample is also determined at step 444. For example, in the cell culture sample, reads that match sequences from contaminants would indicate the presence of that contaminant species in the sample. By analyzing the frequency, type of these matches and UMI abundance, the taxa existing in the sample is identified with high resolution. Example databases can initially include a wide range of genomes from various types of microorganisms, such as bacteria, fungi, virus, and mycoplasma. These genomes are sourced from available public or private commercial databases and can also include organisms specified in relevant regulatory guidelines or industry standards.

At step 430 an estimation model (e.g., a Bayesian estimation or other suitable statistical model) is used to estimate the abundance of different taxa in the sample. At step 446 an estimation model (e.g., Bayesian estimation or other suitable statistical model) is used to estimate the abundance of different taxa in the control. For example, Bayesian models are employed to estimate the abundance of different taxa based on the number and type of sequence matches of the sample and the control. The Bayesian approach uses prior knowledge, such as the phylogenetic relationship between taxa and the likelihood of observing particular taxa in similar environments, along with the new data to update the estimates. For example, in a cell culture sample and/or a control, if a contaminant produces reads which match to several phylogenetically similar taxa, a Bayesian model can be used to estimate the true identity and proportion of that contaminant in the total contaminant community.

At step 432, the quantified taxa abundance of the sample is estimated by the model of step 430. At step 448, the quantified taxa abundance of the control is estimated by the model of step 446. For example, the model computes a probability distribution for the abundance of each taxon, offering a measure of uncertainty along with the estimate. This method can provide a robust statistical basis for inferring the composition of biological communities in the sample.

At step 434, the differential taxa abundance is determined based on the quantified taxa abundance of the sample 432 and the quantified taxa abundance of the control 448. A differential taxa abundance-based statistical testing method can, for example, be used to compare the microbial abundance profiles between the test sample and the negative control. The microbial read abundance profile, in terms of number of reads of each microorganism, is compared between the sample and negative control using a statistical test based on a negative binomial generalized linear model.

In some embodiments, statistical tests such as t-tests or specialized software designed for differential abundance analysis are employed to identify significant differences in taxa levels between the two groups. For example, if a bacterial species is found to be more abundant in a cell culture sample from therapy development and/or manufacture compared to a control cell culture sample, it could be determined to be differentially abundant.

At step 436, significantly present taxa are determined using statistical tests that evaluate the difference in abundance between the control and sample groups. Significantly present taxa, for example, are determined based on the quantified contaminant taxa abundance present in the amplified contaminant nucleic acid sample and the quantified contaminant taxa abundance present in the negative control that determine the differential abundance.

An example metric for determining significantly present taxa is the p-value, which indicates the probability that the observed difference in abundance could have occurred by random chance. A low p-value (e.g., below 0.05) suggests that the difference in taxa abundance is statistically significant. In addition to p-values, false discovery rate (FDR) correction methods can be applied to account for multiple comparisons when many taxa are being evaluated simultaneously. Therefore, taxa with low p-values and adjusted FDR are considered significantly present and warrant further investigation.

FIG. 4B is a flow chart of an example of a distribution model-based workflow for detecting contaminants during therapy development and/or manufacture, as described herein. The example workflow of FIG. 4B, in some embodiments, can include one or more steps of the bioinformatics workflow 242 of FIG. 2. The bioinformatics workflow of FIG. 4B describes a distribution model-based statistical testing method that is used to determine the detection of contaminants from the microbial abundance profiles of a test sample without one or more negative control.

At step 426 the NGS reads of the sample can be obtained. The NGS high-throughput sequencing produces a large number of reads. For example, the sample can be a cell culture sample and the reads produced can include sequences representing the transcripts of contaminants present in the cell culture. In some embodiments, the reads are about 75 nucleotide bases to about 1000 bases in length, e.g., 100, 150, 200, 250, 300, 350, 400, 450, or 500 bases in length depending on the sequencing platform used. The reads can include coding regions, non-coding regions, and any mutations or variations specific to the contaminants. Reads can be either single or paired-end reads. At step 427, the reads are analyzed for taxonomic classification. The resulting NGS reads undergo quality control to remove low-quality or ambiguous sequences. Further filtering or masking of reads may occur based upon reads containing the gene-specific primer sequences and proper location-specific sequence adjacent to the priming location. These cleaned reads can be aligned at step 428 against a reference database that contains known sequences from various taxa, using statistical testing designed for sequence matching. The abundance of UMI for each taxa is also determined at step 428. For example, if the focus is on bacterial DNA, each read that matches a particular bacterial species contributes to the evidence supporting the presence of this species in the sample. The frequency, type of matches, and UMI abundance can be used for the identification of organisms present. For example, for a cell culture sample, reads that match sequences from contaminants would indicate the presence of that contaminant species in the sample. By analyzing the frequency and type of these matches, the taxa existing in the sample is identified with high resolution. Example databases can initially include a wide range of genomes from various types of microorganisms, such as bacteria, fungi, virus, and mycoplasma. These genomes are sourced from available public or private commercial databases and can also include organisms specified in relevant regulatory guidelines or industry standards.

At step 430 an estimation model (e.g., Bayesian estimation or other suitable statistical model) is used to estimate the abundance of different taxa in the sample. For example, Bayesian models are employed to estimate the abundance of different taxa based on the number and type of sequence matches of the sample and the control. The Bayesian approach uses prior knowledge, such as the phylogenetic relationship between taxa and the likelihood of observing particular taxa in similar environments, along with the new data to update the estimates. For example, in a cell culture sample, if a contaminant produces reads which match to several phylogenetically similar taxa, a Bayesian model can be used to estimate the true identity and proportion of that contaminant in the total contaminant community.

At step 432, the quantified taxa abundance of the sample is estimated by the model of step 430. At step 448, the quantified taxa abundance of the control is estimated by the model of step 446. For example, the model computes a probability distribution for the abundance of each taxon, offering a measure of uncertainty along with the estimate. This method can provide a robust statistical basis for inferring the composition of biological communities in the sample.

At step 434, a distribution model is built based on the quantified taxa abundance to distinguish the contaminant from the background. For example, inter-sample (non-negative control compared) machine learning statistical models can be employed, leveraging the cumulative distribution of UMI abundance and/or synthetically derived probability model of observed outcomes, Z-score significance can be determined. These models can determine the threshold that refine the determination of a taxa detection call. In some embodiments, statistical tests such as t-tests or specialized software designed for machine learning are employed to determine the threshold of contaminant identification. For example, if a bacterial genus/species deviates significantly from background genera/species abundance, the bacterial genus/species can be determined as a true contaminant. At step 436, significantly present taxa are determined using statistical tests. Significantly present taxa, for example, is determined based on the cumulative distribution of quantified taxa abundance. An example metric for determining significantly present taxa is the p-value, which indicates the probability that the observed difference in abundance could have occurred by random chance. A low p-value (e.g., below 0.05) suggests that the difference in taxa abundance is statistically significant. In addition to p-values, FDR correction methods can be used to account for multiple comparisons when many taxa are being evaluated simultaneously. Therefore, taxa with low p-values and adjusted FDR are considered significantly present and warrant further investigation.

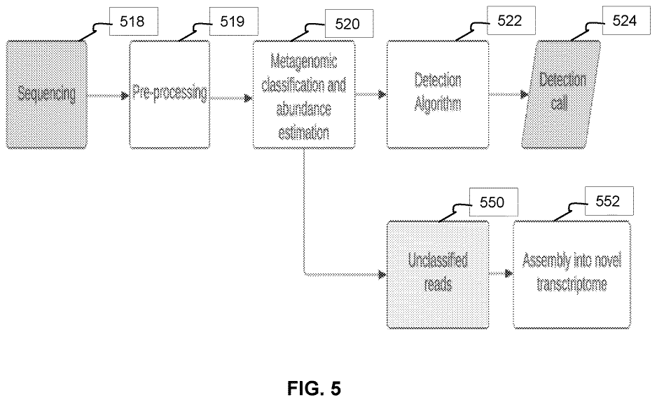

FIG. 5 is a flow chart of an example of a bioinformatics workflow for detecting contaminants during therapy development and/or manufacture, as described herein. FIG. 5 includes an example of an optional workflow to identify unclassified contaminants through transcriptome assembly.

At step 518, the workflow includes sequencing the sample (e.g., NGS) to produce reads. At step 519, the reads generated from the sequencing of the cell culture sample can go through a quality control process for pre-processing. This can involve the separation of low-quality or ambiguous sequences, resulting in a set of pre-processed or cleaned reads.

At step 520, the pre-processed reads are aligned to a reference database that contains sequences of known microorganisms. Specialized algorithms perform this sequence matching to classify the reads into different taxa. In the development of a therapy, contaminant reads that match known contaminant sequences can be flagged for further investigation. Following classification, Bayesian estimation techniques can be applied to quantify the abundance of each identified taxon in the cell culture sample. These estimates are based on the number of reads matching each taxon in the reference database. For instance, if the cell culture sample shows multiple reads matching a particular bacterial species, Bayesian models could estimate the proportion of that bacterial species within the microbial community.

At step 522, a detection algorithm is used to detect differentially abundant taxa. For example, statistical tests, such as t-tests or machine learning models, can be used to detect differentially abundant taxa between the cell culture sample and a control sample (as described in connection with FIG. 4). For example, if a specific contaminant species is found in higher abundance in the cell culture compared to a control, it could be a signal of contamination.

At step 524, a detection call is made for the differentially abundant taxa. For example, statistical measures like p-values and false discovery rates are calculated. If a particular taxon shows significantly different abundance with low p-values and adjusted FDR, it is considered a positive detection call. For example, if bacterial reads are significantly more abundant in the cell culture than in the control, a positive detection call for bacterial contamination is made.

At step 550, unclassified reads can be identified. For example, during the alignment process, some reads may not match any sequences in the reference database. These are categorized as unclassified reads. For example, these could represent microorganisms not included in the existing database. In some examples, the unclassified reads could represent novel or rare microorganisms not included in the existing database.

At step 552, the unclassified reads can be assembled into a novel transcriptome. For example, unclassified reads are assembled into longer sequences called contigs. Contigs can serve for the detection of novel or rare microorganisms not present in the existing databases. For example, a sufficient number of high-quality contig sequences that do not match any known bacterial species could indicate the presence of a novel bacterial strain in the cell culture, potentially making the assay more robust.

### Systems

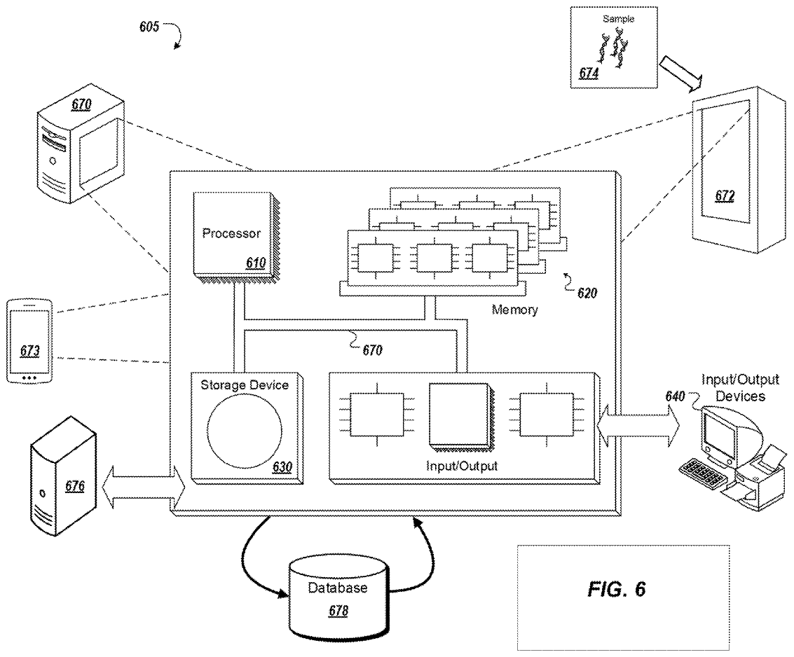

FIG. 6 is a schematic diagram illustrating an example of a computing system 605 used for detecting contaminants that may be present during the development and/or manufacture of cell-based and/or gene-based therapeutic compositions. The computing system 605 can include a hardware device 672 to execute one or more of the workflows described in connection with FIGS. 1-5, one or more computing device 670, and optionally one or more mobile computing devices 673 that can be used to implement the techniques described herein.

The hardware device 672 can manipulate reagents to execute the workflows described in connection with FIGS. 1-5. For example, the hardware device 672 can be equipped with software accessible to users through a user-interface. The reagents for the wet lab workflow can be either preloaded in the device or available in compatible cartridges.

The hardware device 672 can include a port or external access to provide a sample 674 and/or a control to execute the wet lab workflow. Reagents can be included in the hardware device 672 or in compatible cartridges for one or more tasks as described in connection with FIGS. 1-5. For example, reagents for cell depletion, reagents for removal of free-flowing nucleic acids, reagents for lysis, reagents for RNA purification, reagents for human RNA depletion, reagents for cDNA amplification of contaminants, reagents for RNA library construction, and/or reagents for library quality control (QC). In some embodiments, the sequencing will be executed external to the hardware device 672 by the sequencer 676 that can be communicatively coupled to the system 605.

For example, the amplified contaminant nucleic acid sample generated by the hardware device 672 can be removed and sequenced in a sequencer 676 to produce a plurality of reads. The reads generated by sequencing the sample 674 and/or the control by the sequencer 676 can be imported via one or more computers, to the hardware device 672 for a bioinformatics workflow as described in connection with FIGS. 1-5.

The system 605 can include one or more processors 610, one or more memories 620, one or more storage devices 630, and one or more input/output (I/O) devices 640. The components 610, 620, 630, 640 can be interconnected using a system bus 670.

The processor 610 can be configured to execute instructions within the system 605. For example, the processor 610 can execute instructions for the methods described herein (e.g., the example method of FIG. 1). The processor 610 can include a single-threaded processor or a multi-threaded processor. The processor 610 can be configured to execute or otherwise process instructions stored in one or both of the memory 620 or the storage device 630. Execution of the instruction(s) can cause graphical information to be displayed or otherwise presented via a user interface on the I/O device 640.

The memory 620 can store information within the system 605. In some implementations, the memory 620 is a computer-readable medium. In some implementations, the memory 620 can include one or more volatile memory units. In some implementations, the memory 620 can include one or more non-volatile memory units.

The storage device 630 can be configured to provide mass storage for the system 605. For example, the storage device 630 can store a database of reference sequences. In other examples, a database 678 that is stored external to the computer system 670. In other embodiments, the database 678 can be a part of computer system 670.

In some implementations, the storage device 630 is a computer-readable medium. The storage device 630 can include a floppy disk device, a hard disk device, an optical disk device, a tape device, or other type of storage device. The I/O device 640 can provide I/O operations for the system 605. In some implementations, the I/O device 640 can include a keyboard, a pointing device, or other devices for data input. In some implementations, the I/O device 640 can include output devices such as a display unit for displaying graphical user interfaces or other types of user interfaces.

### Kits

This disclosure also includes kits. For example, a hardware device can be equipped with software accessible to users through a user-interface. The hardware device can be preloaded with reagents. In some embodiments, the workflows can be customized and cartridges compatible with the hardware device can be provided based on specification provided by a user. For example, one or more cartridges can be customized based on the cell-based and/or gene-based therapeutic compositions being developed and/or manufactured. In some embodiments, the cartridges can include reagents used in the methods described above. In some embodiments, the reagents used in sequencing are omitted from the kit and/or cartridge.

### EXAMPLES

The disclosure is further described in the following examples, which do not limit the scope of the disclosure described in the claims.

The following examples are an evaluation of the efficacy of assays described herein that are used to increase contamination signal detected via NGS to determine one or more contaminants at the genus and/or species level.

### Example 1: Enrichment of Mycoplasma Cells in the Background of Human Jurkat Cells To Demonstrate Human Cell Depletion

Jurkat cells contaminated with mycoplasma were maintained in culture conditions. To facilitate Mycoplasma enrichment, an antibody-based depletion strategy was applied. Antibodies specific for CD3-positive and non-T-cell peripheral blood mononuclear cells (PBMC) markers were used to selectively deplete human Jurkat cells from the culture. Post-depletion, the cell samples were subjected to NGS sequencing to evaluate mycoplasma read alignment. This data was compared against a No Selection control to assess the effectiveness of the depletion strategy in enriching mycoplasma cells. A t-test was employed to determine the statistical significance of the fold-change in mycoplasma read alignment. Results with a p-value less than 0.05 were considered significant.

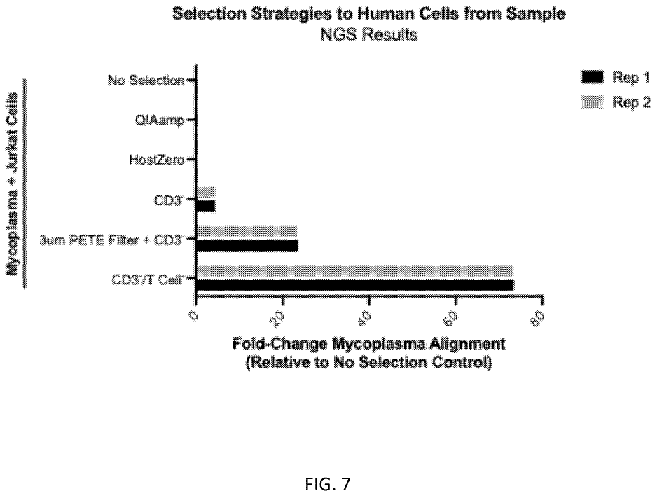

The results are shown in FIG. 7, which shows a comparison of methods to selectively deplete background human cell signal, thereby enriching the biological contaminant signal (Mycoplasma). Three depletion methods: 1) “CD3-positive” antibody selection, 2) “3 micron filtration with CD3-positive”, and 3) “CD3-positive and non-T cell-marker positive,” all showed an enrichment of mycoplasma signal using a qPCR assay readout as compared with No Selection, QIAamp, and HostZero. CD3-positive and non-T-cell marker positive samples showed the most pronounced enrichment of mycoplasma signal over “no selection” controls. This shows an improved ability to find low-abundance contamination in cell therapy products. This approach can be extended to depletion of high-prevalence background cell types using an antibody-based approach.

### Example 2: Evaluation of Human Nucleic Acid Depletion

The aim of this experiment was to enrich mycoplasma cells in a background of human Jurkat cells by depleting human rRNA sequences.

Nucleic Acid Depletion: Selective ribosomal depletion was performed using commercially available reagents specifically targeting human rRNA sequences. This approach achieved greater than 95% depletion of human rRNA without affecting the proportion of contaminating Mycoplasma rRNA reads.

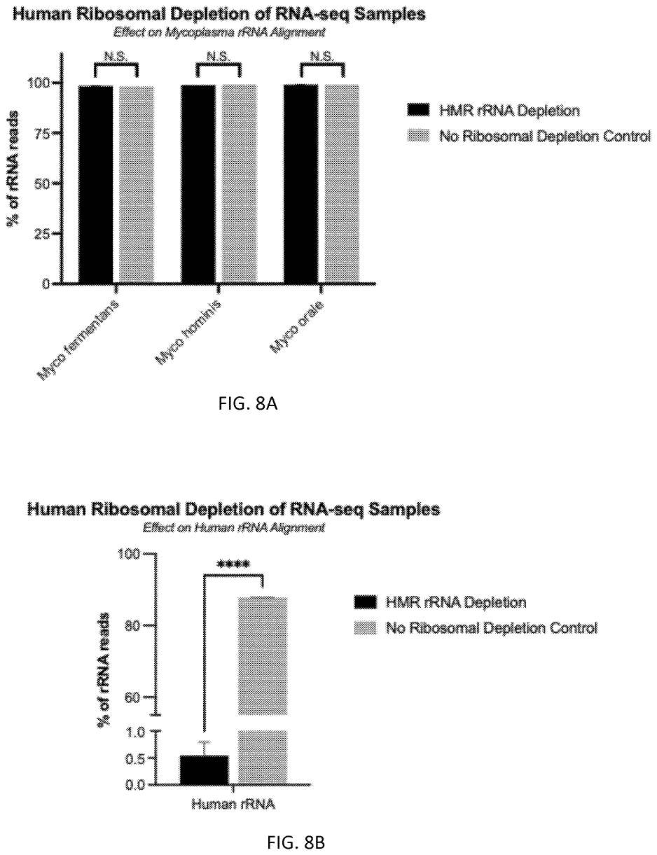

RNA Expression Signature: In addition to generic rRNA depletion, a unique RNA expression signature was developed from a CAR T-cell product. Human genes contributing to more than 20% of the total reads were selected. Targeting exon 5 of the human beta-actin gene effectively reduced its transcript signal. Sensitivity Assessment: Assay sensitivity was evaluated by spiking in Mycoplasma cells at levels ranging from 1000 to 10,000 CFU in 50×10⁶ Jurkat cells (See FIG. 8C) where (?) indicates mapped over reads, (†) indicates mapped human reads, and (‡) indicates mapped Mycoplasma reads. Mycoplasma enrichment was confirmed through NGS (FIGS. 8A-8D). Statistical Analysis: A t-test was used for determining the statistical significance of the changes in Mycoplasma read alignment. A p-value of less than 0.05 was considered significant.

To increase the detection sensitivity of biological contaminants, apart from depletion of human cells (e.g., FIG. 7), even a small number of human cells coming through antibody depletion (e.g., FIG. 7), will overwhelm contaminant signal in the NGS prep. To eliminate any residual, but nevertheless substantial amount of human signal, depletion of human nucleic acid from partaking in NGS library prep. was established. This step is done after cell depletion in FIG. 7, as steps in FIG. 8 rely on access to extracted nucleic acid. The extracted nucleic acid is over 90% if human RNA is ribosomal in nature.

FIG. 8A shows that methods of depletion of human ribosomal RNA do not deplete signal from three distinct species of mycoplasma. FIG. 8B addresses the efficiency of the depletion kit in reducing signal from human nucleic acid-which is the desired outcome. FIG. 8C is a demonstration that employing approaches of ribosomal human depletion (as shown in FIGS. 8A and 8B), mycoplasma signal was found in NGS data, even when the initial sample had 50 million human Jurkat T-cells. Given the vast excess of human material/mycoplasma biological contamination ratio, this would have been unlikely without human ribosomal depletion steps being included in the workflow.

FIG. 8D is another bar graph that shows the results of further suppressing human signal by LNA blocking oligos that were used to specifically bind to highly expressed human transcripts (beyond human ribosomal transcripts). FIG. 8D shows utility of using custom LNA blocking oligonucleotides that block exon 5 transcripts of the human B-actin gene, and the exons that are 5′ of this exon (exons 1 through 5). Strong suppression of exon 5 NGS data was determined and exon 6 is not as strongly inhibited—this is expected because exon 6 is 3′ of the LNA primer and transcription goes 5′ to 3′ (thus not inhibiting reverse transcription of exon 6).

### Example 3: Evaluation of Gene Specific Amplification of Adventitious Agents

The aim of this experiment was to enhance the detection of specific bacterial and fungal contaminants within a background of human cells by utilizing gene-specific priming techniques in next-generation sequencing assays.

Gene-Specific (GS) Amplification of Adventitious Agents: Primers were designed to target conserved ribosomal regions (16S, 18S, 25S, ITS1, and 28S) in specific bacterial and fungal genomes, including (*) Mycoplasma fermentans, (†) Staphylococcus aureus, (‡) Aspergillus brasiliensis, (+) Pseudomonas aeruginosa, (=) Candida albicans, and (@) Mycoplasma hominis.These primers were utilized during the first-strand cDNA synthesis phase to selectively amplify non-human, contaminating sequences. In addition, random hexamers can be used alongside these primers to capture organisms not covered by the gene-specific primers. This strategy increased the number of contaminating reads and simultaneously reduced the number of human reads.

Statistical Analysis: A t-test was employed to assess the statistical significance of the changes in read alignment. Results with a p-value of less than 0.05 were considered significant.

Biological contaminants such as bacterial and fungal species have unique ribosomal RNA sequences (e.g., 16S, 18S, ITS 1, and 25S ribosomal RNA). These sequences are not present with high sequence homology in humans. Therefore, to specifically enrich for biological contaminant signal, reverse transcription gene-specific primers were designed to these ribosomal motifs that bind to relevant biological contaminants, initiating cDNA synthesis of contaminating microorganism RNA to ultimately make an NGS library. By contrast, commonly used methods such as random priming of RNA do not discriminate conversion of RNA into an NGS library between species and will prime from human RNA as well as contaminating microorganisms, thereby reducing sensitivity of contaminating microorganism detection.

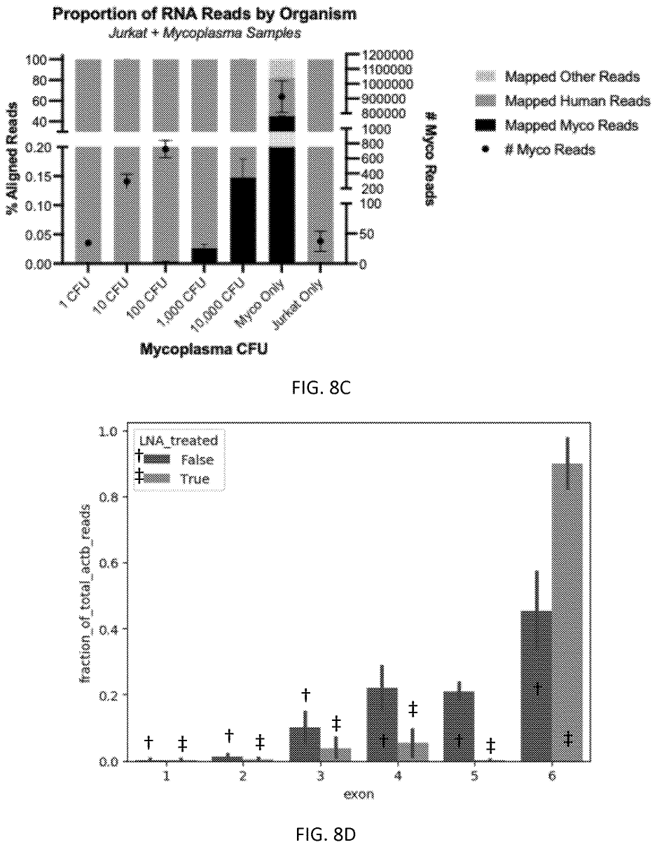

FIG. 9A shows that for multiple contaminants, an enhanced signal from various contaminating microorganisms were identified, as compared to when using the more typical random priming strategy. This shows that the methods described herein are selectively generating more meaningful signal from the contaminating microorganism using gene-specific priming.

FIG. 9B is another bar graph that shows the increased detection sensitivity of contaminating microorganisms in FIG. 9A is partly because of reduced priming of human RNA in the GS condition (see first bar on graph). The 4^{th} bar of the graph is no different from bars 5 and 6. This helps solidify/prove the hypothesis that the key reason for lower human signal in GS conditions is due to contamination sequences being better represented at the expense of human reads taking up sequencing data.

### Example 4: Evaluation of Chemical Reduction of Signal Originating From Dead Cells And Contaminating DNA

The aim of this experiment was to minimize background noise in RNA-seq data by selectively reducing signals originating from dead cells and contaminating DNA sequences.

Chemical Reduction of Signal Originating from Dead Cells and Contaminating DNA: Intercalating dyes such as platinum chloride, propidium monoazide, and ethidium monoazide were used to chelate free-floating nucleic acids and inhibit their amplification. Platinum compounds specifically enabled discrimination between live and dead microorganisms. This approach reduced the signal from free-floating Escherichia coli DNA in a human T-cell background after treatment with platinum chloride.

Statistical Analysis: Significance of reductions in background noise was assessed using a t-test. A p-value of less than 0.05 was deemed significant.

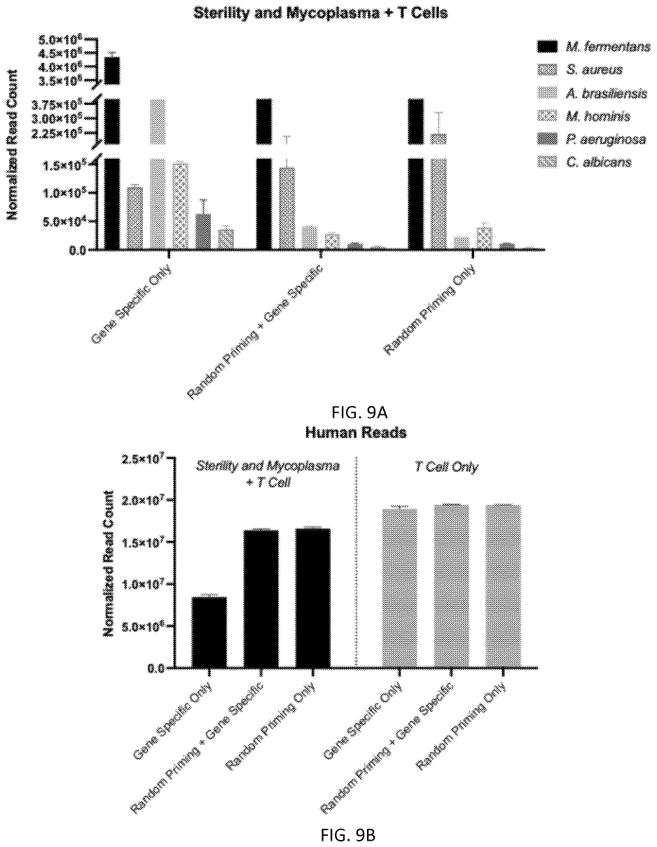

FIG. 10 is a bar graph that shows experimental measurements, which suggest that dying human cells release DNA into the sample. DNA is particularly problematic as it is very stable and will not degrade easily. Therefore, DNA can persist in downstream steps of the NGS library preparation (intergenic reads have been observed in our NGS data that originate from DNA). DNA contamination of human cells is problematic as it may overwhelm the NGS signal.

Bacterial DNA contamination is also observed in reagents used for NGS preps (e.g., in enzyme preps) and this could incorrectly be assigned to presence of contaminating microorganisms (even in an RNA assay). This type of DNA can hinder assay sensitivity. We used chemical agents, like platinum chloride, to degrade free-floating DNA. To test this, E. coli DNA was added into the prep, in the presence and absence of DNA-degrading platinum chloride. The first pair of bars in the bar-graph († and +) show a substantial reduction in E. coli readout when platinum chloride is added to the reaction. This shows promise for platinum chloride to reduce background signal contamination coming from DNA coming from dying human and contaminating microorganisms, as well as DNA contamination in reagents used for our assay.

The platinum chloride must be added to the reaction before cell lysis, otherwise all nucleic acids (DNA and RNA) from biological contaminants will be degraded by platinum chloride or similar agents. In addition (or alternatively), enzymatic approaches for DNA removal, such as treatment with DNase, can be added after cell lysis to destroy all residual human and contaminant DNA. These treatments are aimed at generating a pure RNA sample for subsequent NGS library preparation steps, thereby enriching for live microorganism contaminants in the cell culture sample.

Further in FIG. 10, the second set of bars only has one bar, and the “missing” bar is for the treated sample, in which the treatment blocks free-floating E. coli nucleic acid from becoming a library that can be sequenced. The bar that is visible is lower than the corresponding bar in the first set, because the input E. coli nucleic acid is present at a level of 0.35 ng, which ten-fold lower than the amount of 3.50 ng E. coli represented in the first pair of bars. The third pair of bars are also “missing,” because no E. coli nucleic acid was added to these samples.

### Example 5: Evaluation of a Process Control

The aim of this experiment was to implement robust control strategies for ensuring the sterility and performance of NGS assays, which includes reducing background noise and real-time assay validation.

Process Control: For quantification and sensitivity qualification, unique RNA spike-ins were developed. These synthetic RNA fragments, distinguishable from both human and microbial sequences, contain unique and non-overlapping barcodes for retrieval during analysis. These spike-ins were incorporated at a copy number higher than the assay's limit of detection after cell lysis but before sample and library preparation. By analyzing the linear relationship between the input concentration of these RNA spike-ins and the retrieved read frequencies, the assay's performance was confirmed on a run-by-run basis. Deviation from this linear relationship would warrant rejection of the assay results. Statistical Analysis: A linear relationship between the input concentration of the process control RNA and the retrieved read frequencies is expected for valid assay results.

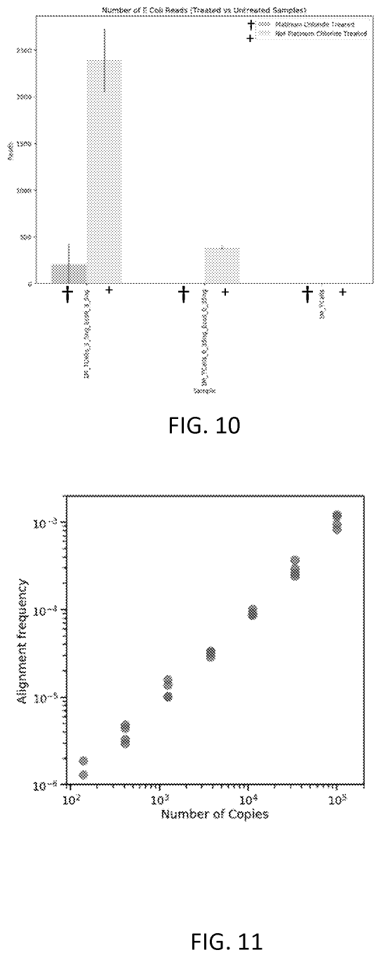

FIG. 11 shows absolute quantification and sensitivity qualification of the assay. A set of RNA spike-ins that have unique properties that make them relevant for our assay compared with commonly used RNA spike-ins, such as External RNA Controls Consortium (ERCC) spike-ins. This positive control consists of a synthetic RNA, with a sequence structure that is distinguishable from both human and microbial sequences. In FIG. 11, spike-in controls are detected in NGS readouts in a linear response to initial input copy number, which indicates the spike-in NGS readout is linear and quantitative. Additionally, the readout is highly robust as seen by technical replicate reproducibility.

### Example 6: Evaluation of the Effectiveness of a Negative Control in Reducing Background

The aim of this experiment was to demonstrate the effectiveness of the statistical testing (differential abundance) algorithm in distinguishing true signal (*) from background noise (+).

Integrated Metagenomic and Gene Expression Analysis Workflow: In this study, a bioinformatics workflow was employed that utilized metagenomic tools for the taxonomic classification of NGS reads and estimation of their abundance. RNA-seq was used for the quantitative analysis of the transcriptome as described above with respect to FIGS. 4-5. The bioinformatics pipeline initially performed taxonomic classification against a microbial genome database. Bayesian methods were then utilized for abundance estimation of each discovered taxa. The abundance of these taxa in the test sample was compared to their abundance in a negative control using differential abundance analysis.

Sensitivity Challenges and Negative Control in NGS-Based Assays: Due to the high sensitivity of NGS assays, trace microbial reads from environmental or reagent sources could lead to false positives. To counter this, a negative control sample was processed and sequenced along with the test samples. This control allowed the algorithm to discriminate between true microbial presence and external contamination.

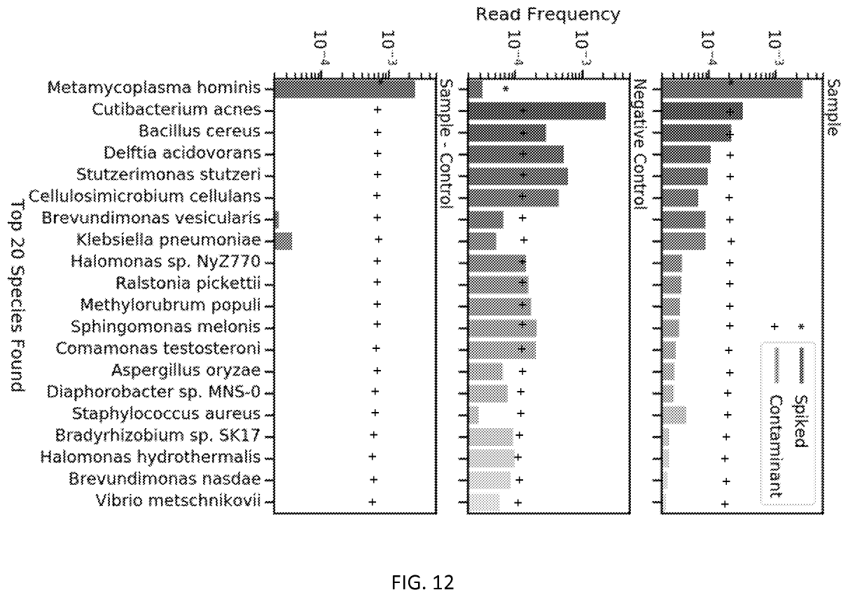

Statistical Analysis: Referring to FIG. 12, the top pane is a bar graph that shows the initial microbial counts in the sample (spiked vs contaminants). The middle pane is a second bar graph that shows microbial counts from the negative control (spiked v contaminants). Differential abundance analysis was conducted to compare these two data sets. The bottom pane is a third bar graph that presents the adjusted microbial counts, effectively separating true signals from background noise.

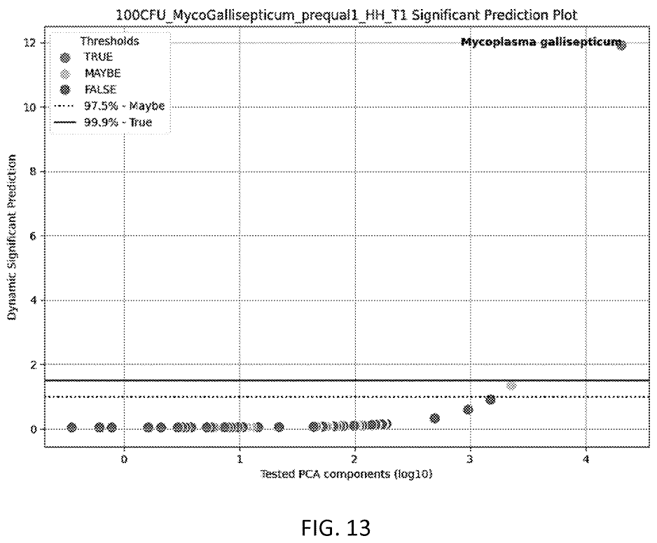

If no negative controls are provided, machine learning of the cumulative distribution of detected taxa can be used to differentiate the true contaminant and the background taxa as shown in FIG. 13. The thresholds of true contaminants (TRUE), potential contaminants (MAYBE), and background (FALSE) were determined dynamically for each sample based on the abundance distribution (UMI for example) of detected taxa using a robust Z-score statistic.

As shown in FIG. 13, the y-axis shows machine learning determined significance calculated based on the robust Z-score of cumulative counts (including, but not limited to, using an algorithm that determines an estimated true UMI count, read counts, or normalized read counts). The x-axis shows the rescaled or linearized results using dimension reduction from one type or multiple types of cumulative counts (e.g., estimated true UMI, unique UMI counts, read counts, or normalized read counts). In this example, Mycoplasma gallisepticum was spiked into the cell culture sample. Among all of the microorganisms detected, the machine learning method identified one “TRUE” contaminant above the true contaminant threshold, Mycoplasma gallisepticum, as expected (see the data point in the far upper right corner of the graph). One potential contaminant is Mycoplasma imitans, which is in the same genus as M. gallisepticum and shares genetic similarities. The rest of the species all contribute to background noise. In addition, the graph in FIG. 13 shows only one “MAYBE” point (light gray) just under the “true line” at about the 1.5 mark between 3 and 4 on the x axis, and the rest of the data points along the bottom are all “FALSE” readings, all as expected, which confirms the utility of the methods described herein.

### Other Embodiments

It is to be understood that the foregoing description is intended to illustrate and not limit the scope of the invention, which is defined by the scope of the appended claims. Other aspects, advantages, and modifications are within the scope of the following claims.

What is claimed is:

1. A method for detecting contaminants during manufacture of a therapeutic composition, the method comprising:
(a) obtaining a sample from a liquid used in the manufacture of the therapeutic composition, wherein the sample comprises a plurality of production cells and a potential contaminant;
(b) removing a production cell of the plurality of production cells from the sample to generate a production cell-depleted sample;
(c) selectively reducing a level of production cell nucleic acids from the production cell-depleted sample to produce a contaminant nucleic acid-enriched sample;
(d) amplifying contaminant nucleic acids in the contaminant nucleic acid-enriched sample using a nucleic acid primer that selectively hybridizes with a conserved genomic nucleic acid sequence region of the potential contaminant to produce an amplified contaminant nucleic acid sample;
(e) sequencing the amplified contaminant nucleic acid sample to produce a plurality of reads; and
(f) determining, using an abundance estimation, a quantified contaminant taxa abundance profile of the amplified contaminant nucleic acid sample.

2. The method of claim 1, wherein the contaminant nucleic acid-enriched sample comprises RNA.

3. The method of claim 1, wherein the production cells comprise mammalian cells.

4. The method of claim 1, wherein the production cell is removed in (b) based on a cell surface marker of the production cell, a size of the production cell, a density of the production cell, or a combination thereof.

5. The method of claim 4, wherein the cell surface marker comprises CD3, CD4, CD8, CD28, CD41, CD45, CD62, CD138, CD235a, CD14, CD123, PD-1, ID1, CTLA-4, CD 19, CD20, CD22, CD56, CD 16, CD 14, CD 15, CD66, CD34, or a combination thereof.

6. The method of claim 1, wherein the selectively reducing the level of the production cell nucleic acids in (c) comprises removing at least 90% of the production cell nucleic acids.

7. The method of claim 1, wherein the potential contaminant comprises a fungus, a yeast, a bacterium, a virus, a mycoplasma, or a combination thereof.

8. The method of claim 1, wherein the sample is a cell culture sample, and wherein the plurality of production cells comprise a plurality of mammalian T-cells.

9. The method of claim 1, wherein prior to step (d), the method further comprises lysing cells remaining in the production cell-depleted sample.

10. The method of claim 1, wherein step (c) comprises inhibiting amplification of a particular gene using a complementary oligonucleotide that prevents reverse transcription of the particular gene.

11. The method of claim 10, wherein the particular gene is a human beta actin gene.

12. The method of claim 10, wherein the particular gene encodes human cytoplasmic rRNA 5S, human cytoplasmic rRNA 5.8S, human cytoplasmic rRNA 18S, human cytoplasmic rRNA 28S, human mitochondrial rRNA 12S, human mitochondrial rRNA 16S, human mitochondrial rDNA NDl, human mitochondrial rDNA ND2, human mitochondrial rDNA ND3, human mitochondrial rDNA ND4, human mitochondrial rDNA ND4L, human mitochondrial rDNA ND5, human mitochondrial rDNA ND6, human mitochondrial rDNA COXI, human mitochondrial rDNA COX2, human mitochondrial rDNA COX3, human mitochondrial rDNA ATP6, human mitochondrial rDNA CYTB, or a combination thereof.

13. The method of claim 1, wherein the conserved genomic nucleic acid sequence region of the potential contaminant comprises a 16S ribosomal region of the potential contaminant, a 18S ribosomal region of the potential contaminant, a 25S ribosomal region of the potential contaminant, a ITS1 ribosomal region of the potential contaminant, a 28S ribosomal region of the potential contaminant, a portion thereof, or a combination thereof.

14. The method of claim 1, wherein step (d) further comprises using a random hexamer with the nucleic acid primer, wherein the random hexamer hybridizes with the conserved genomic nucleic acid sequence region of the potential contaminant.

15. The method of claim 1, wherein prior to step (d), the method further comprises applying an intercalating dye to the sample.

16. The method of claim 1, wherein the abundance estimation is a differential abundance algorithm.

17. The method of claim 1, wherein step (f) further comprises: comparing the plurality of reads from the amplified contaminant nucleic acid sample to a database of microbial genomes; identifying one or more contaminant taxa, based on a comparison of a plurality of reads of the amplified contaminant nucleic acid sample to the database of microbial genomes; sequencing a negative control to produce a plurality of reads from the negative control; comparing the plurality of reads from the negative control to the database of microbial genomes; and identifying one or more contaminant taxa in the sample, based on a comparison of results from the negative control to results from the sample.

18. The method of claim 1, further comprising filtering or masking a read of the plurality of reads.

19. The method of claim 1, wherein prior to (f) the method further comprises: aligning the plurality of reads from the amplified contaminant nucleic acid sample to a database of microbial genomes; determining an alignment quality of respective reads of the plurality of reads of the amplified contaminant nucleic acid sample; and omitting respective reads of the plurality of reads from the amplified contaminant nucleic acid sample when the alignment quality of the respective reads is below a threshold.

20. The method of claim 1, wherein the selectively reducing in (c) comprises contacting the production cell-depleted sample with a platinum-based compound, a palladium-based compound, ethidium monoazide (EMA), propidium monoazide (PMA), or a combination thereof.
